(19) United States
(12) Patent Application Publication
(10) Pub. No.: US 2010/0276322 A1
(43) Pub. Date: Nov. 4, 2010

(54) ICE MASSAGE DEVICE

(76) Inventor: Malgorzata KOTNOWSKA, (US)

Correspondence Address: RABIN & Berdo, PC, 1101 14TH STREET, NW, SUITE 500, WASHINGTON, DC, 20005, US

(21) Appl. No.: 12/810,305

(22) PCT Filed: Oct. 24, 2007
(86) PCT No.: PCT/PL07/00071

§ 371 Date: Jun. 23, 2010

(30) Foreign Application Priority Data
Aug. 13, 2007 (PL) W 116968

(51) Int. Cl. A61F 7/10

(52) U.S. Cl. 206/438

(57) ABSTRACT

The subject of the invention is an ice massage device for body care and rehabilitative purposes. The ice massage device comprising a container [1], the container lid [3], the lid handle [5] and an element [4] fixing ice to the lid is characterised in that the lid [3], the lid handle [5] and the ice-fixing element [4] make one whole, while in the bottom of the uniform, open-at-the-top container [1] there is a cavity [2] in the shape of a cone directed towards the inside of the container.

The subject of the invention is a device for massaging the body with ice for body-care and rehabilitative purposes.

There are know devices used for massaging the body, both manual and mechanical. The ice massage device, due to its construction and simple use will be among simple manual massaging devices such as brushes or gloves. The most commonly known of these is a brush provided with a handle and the massaging surface having massaging appendages instead of bristle.

It was found that the effect of a massage is intensified if an ice surface is used for massaging due to the therapeutic effect of low temperature of the massaging surface, which additionally stimulates the circulatory system. It has a beneficial impact not only on the circulatory system, but also on the lymphatic and respiratory systems and the metabolism.

An ice massage device is know from utility design No. 62349 Ice Massage Device. It consists of a lower, middle and upper parts. The lower part has a number of oval chambers widening towards the top and adjoining each other. The middle part is a lid at the bottom of which a net in the shape of a sphere segment made from intersecting fibres is placed, and on the top the lid has guides of which one has two locking elements. The upper part is formed by a handle in the shape of letter “S”, and at the bottom by fixing elements—longitudinal grooves for the guides. In the upper part there is also located a lock that wedges up in the lid lock notch.

However, during the use of the presented device, it turned out that the division of the container into chambers resulted in the formation of a non-uniform block of ice whose melting time was too short to perform an effective massage, also the flatly set lid of the device was deformed by ice which increased its volume during freezing.

The inventive solution eliminates the above mentioned problems and permits a more effective ice massage while providing a technologically simpler production process.

The essential quality of the ice massage device comprised of a container, the container lid, the lid handle and an element fixing ice to the lid is that the lid, the lid handle and the ice-fixing element make one whole and in the bottom of the uniform, open-at-the-top container a cavity in the shape of a cone directed inwards is formed.

Preferably the container edge and lid are bent in the form of an arch.

Preferably the container edge has a rim protecting the lid against sliding off the container edge.

Preferably the container edge adjoins the rim of the container edge.

Alternatively the container edge and lid are flat.

Preferably the circumference of the edge of the flat lid is smaller than the circumference of the flat edge of the container.

Preferably the container has the shape of a polyhedron with a polygon base, whose walls join the base at an angle of not less than 90°.

Preferably the joints of the container walls are semicircular.

Alternatively the edges of the interconnected walls of the container are sharp.

Alternatively the container has the form of a hemisphere truncated at the bottom.

Alternatively the walls and the base of the container have a stepwise structure consisting of rings that join one another and whose circumferences decrease towards the bottom and which are arranged on a transverse plane, parallel in relation to one another and perpendicularly to the basis.

Preferably the stepwise structure permits the overlapping of the interconnected rings in such way that rings of a smaller circumference are placed inside rings of a bigger circumference, which allows the container to be folded.

Alternatively the container in its cross-section has the shape of a closed figure with an irregular circumference.

Preferably in the bottom on the outside of the container there are formed additional swellings of material in the form of patterns on which the container rests.

Preferably the formed additional swellings of material in the form of patterns are located in the bottom on the outer side and on the inner side of the container.

Alternatively in the swellings of the material made in the container bottom on the outside, there are grooves on the inner side of the container.

Preferably the rim of the edge of the container is deflected outside forming an additional outer wall of the container, on whose edge the container is placed.

Preferably the container supported on the edge of the outer wall has the shape of a hemisphere.

Preferably the element that fixes ice to the lid has the shape of an arched flat bar slightly deformed in the middle.

Preferably the element that fixes ice to the lid is at least double.

Preferably the element that fixes ice to the lid is additionally attached to the lid bottom by appendages.

Alternatively the element that fixes ice to the lid consists of several appendages ending with brims.

Preferably the element that fixes ice to the lid has interconnected brims.

Preferably the element that fixes ice to the lid has openings running across the ice-fixing element.

Preferably the lid handle is attached at its both ends to the top part of the lid.

Alternatively the lid handle is attached at only one its end to the top part of the lid and has the shape of a quarter of an oval.

Preferably the lid handle is at least double.

Alternatively the lid handle is fixed on a core.

Preferably in the lid handle there are openings running across the handle.

An advantage of the inventive device is that it allows to perform the cryotherapeutic ice massage for a long time. This is especially important when applying the ice massage in variable-temperature therapy, that is during a warm bath or shower which accelerate the ice melting process.

The uniform shape of the container without a division into chambers forms a block of ice which is compact and which thereby has a bigger volume and thus a longer melting time, which results in a longer massage time.

An advantage of the device is the use of a simple and functional ice-fixing element in the form of an arched flat bar deformed in the middle of the arc in the opposite direction, which is useful because it anchors ice in a more stable way and increases the proportion of ice quantity in the block used for the massage, which allows the massage to last longer.

The ergonomic broad handle offers a stable and sure grip which facilitates the application of the ice massage and causes that the weight of ice is naturally used to enhance the massage intensity.

An advantage of the inventive device is also its simple construction facilitating the technological production process and ensuring an intuitive use of the device which translates into the frequency of the application of the cryotherapeutic massage.

The subject of the invention is shown in the drawing where

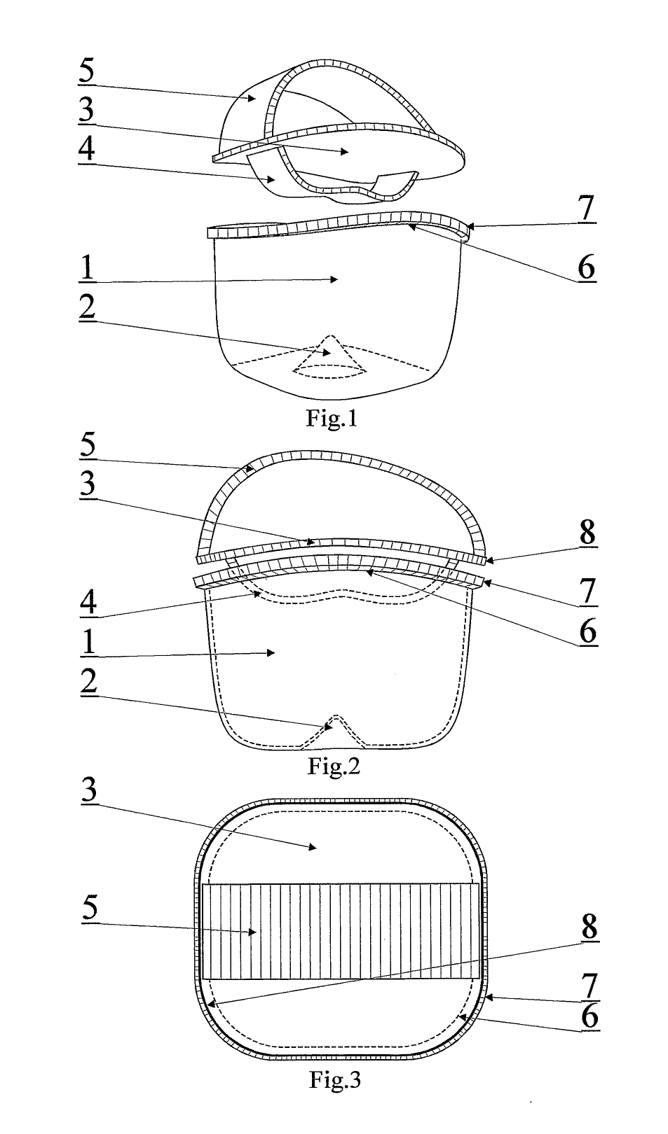

FIG. 1 shows a perspective view of the device,

FIG. 2 a side view of the device in its first embodiment, showing that the lid and the edge of the container are bent in the form of and arc,

FIG. 3 top view of the device shown in FIG. 2 showing that the edge of the lid adjoins the container edge rim which protects the lid against sliding off the container edge,

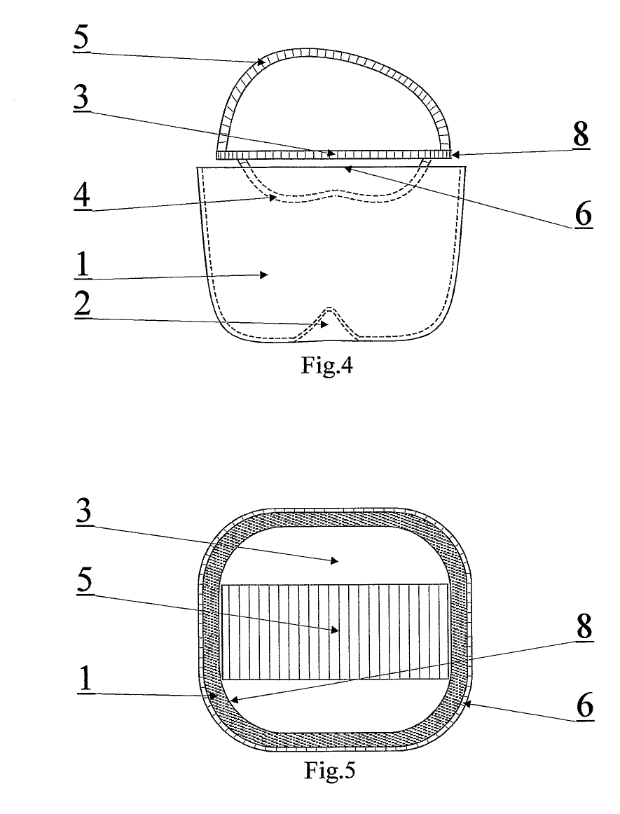

FIG. 4 a side view of the device in its second embodiment, showing that the container lid and edge are flat,

FIG. 5 top view of the device shown in FIG. 4 showing that the lid edge circumference is smaller than the container edge circumference,

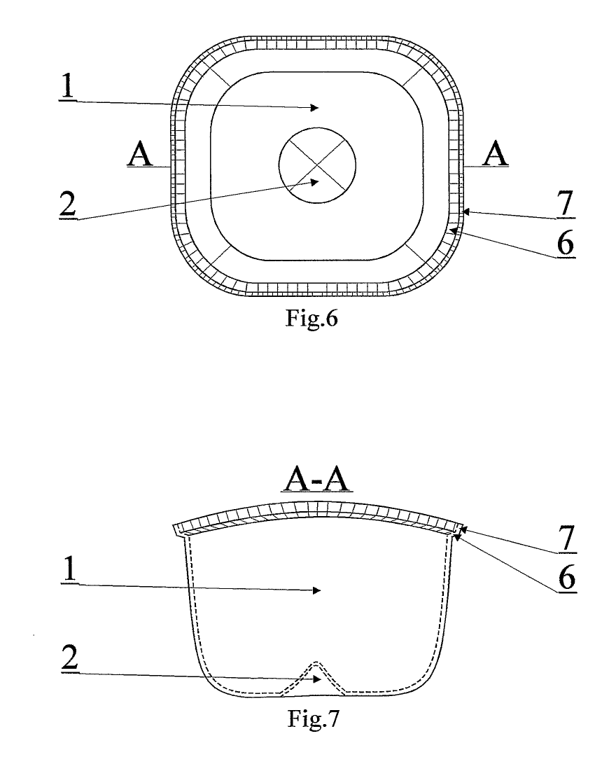

FIG. 6 top view of the container in its first embodiment showing that the joints of the walls are semicircular and that the container edge is finished with a rim,

FIG. 7 section in A-A plane indicated in FIG. 6,

FIG. 8 top view of the container in its second embodiment showing the edge of the container and the semicircular joints of the walls,

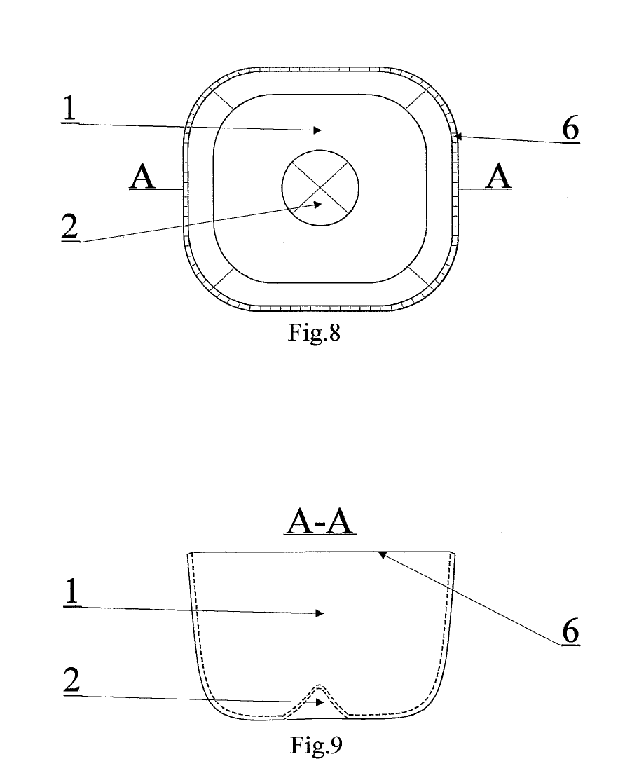

FIG. 9 section in A-A plane indicated in FIG. 8,

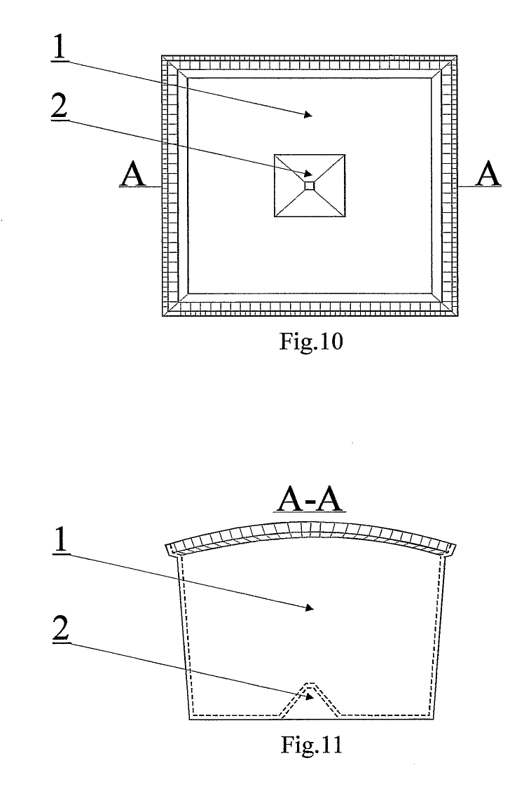

FIG. 10 top view of the container in its third embodiment showing that the edges of the walls are sharp,

FIG. 11 section in A-A plane indicated in FIG. 10,

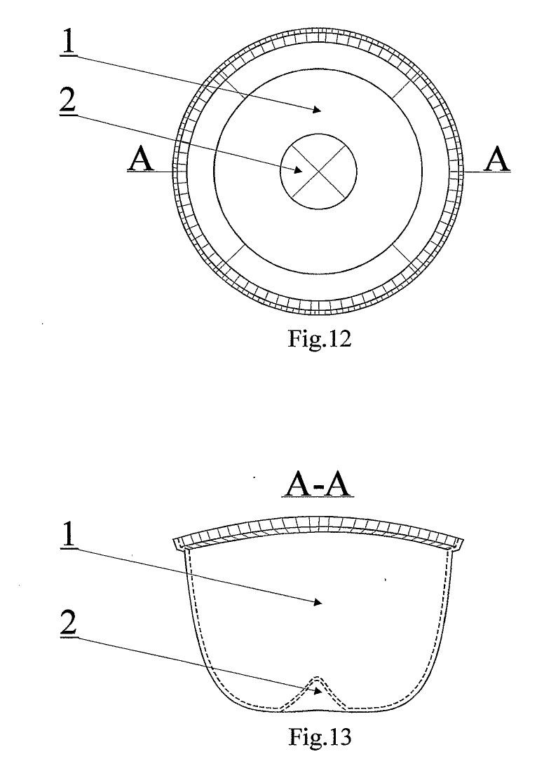

FIG. 12 top view of the container in its fourth embodiment which has the form of a hemisphere truncated at the bottom,

FIG. 13 section in A-A plane indicated in FIG. 12,

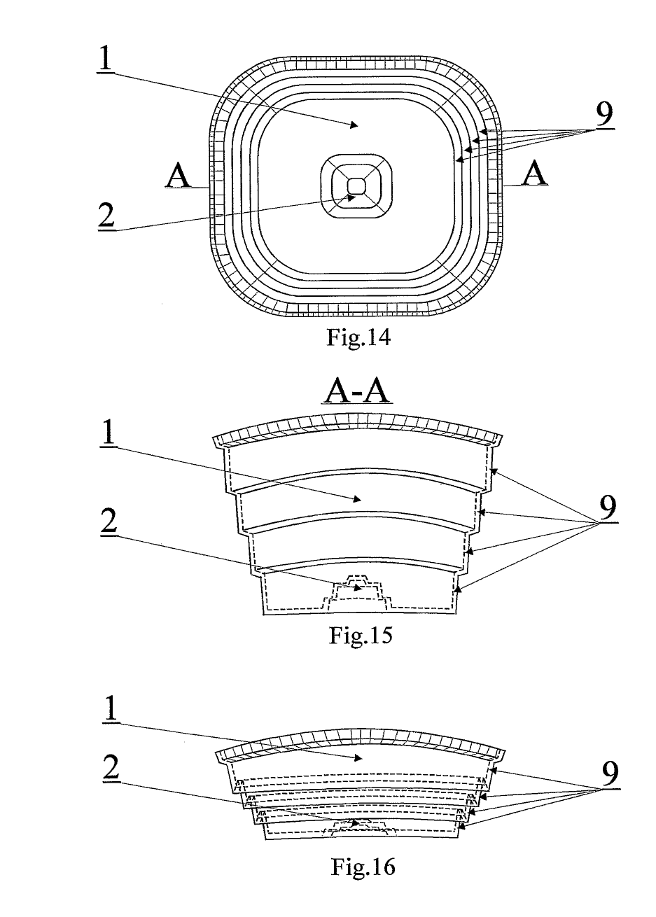

FIG. 14 top view of the container in its fifth embodiment whose walls and base has a stepwise structure,

FIG. 15 section in A-A plane indicated in FIG. 14,

FIG. 16 side view of the container shown in FIG. 14, having a stepwise structure, which is folded so that rings of smaller circumferences are placed inside rings of bigger circumferences,

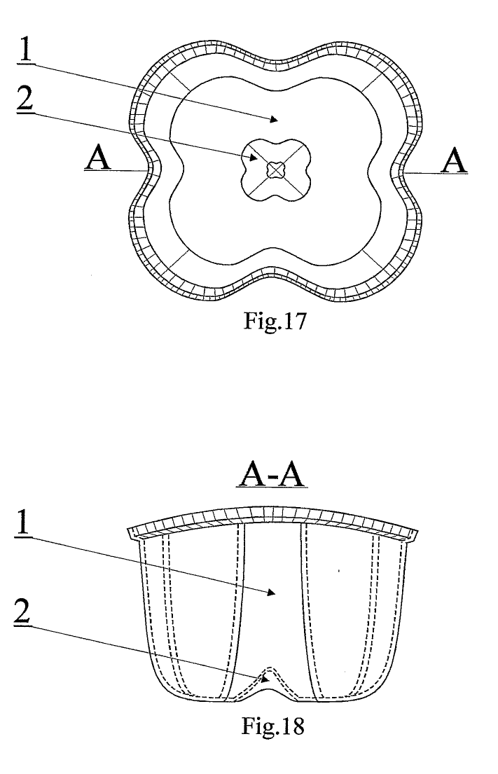

FIG. 17 top view of the container in its sixth embodiment which in its cross-section has the shape of a closed figure with an irregular circumference,

FIG. 18 section in A-A plane indicated in FIG. 17,

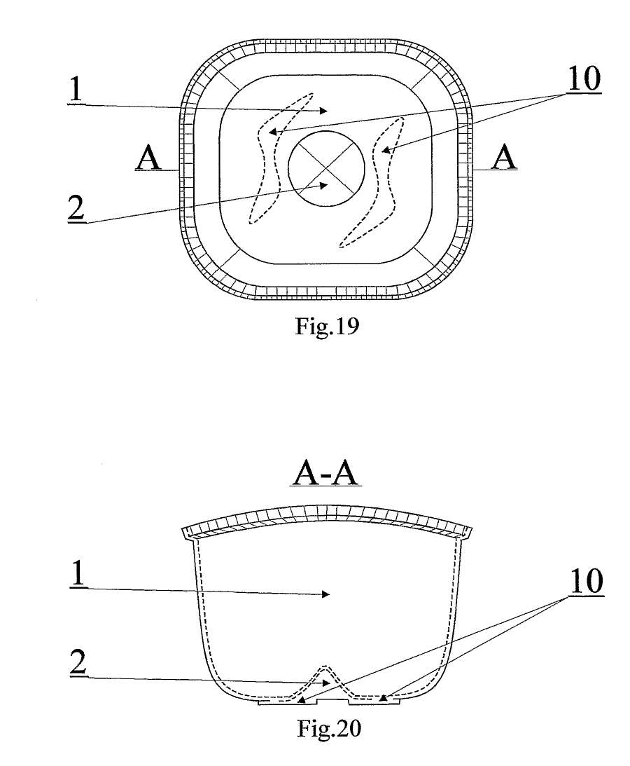

FIG. 19 top view of the container in its seventh embodiment, showing swellings of the material in the form of patterns on which the container rests, additionally formed in the container bottom on its outer side,

FIG. 20 section in A-A plane indicated in FIG. 19,

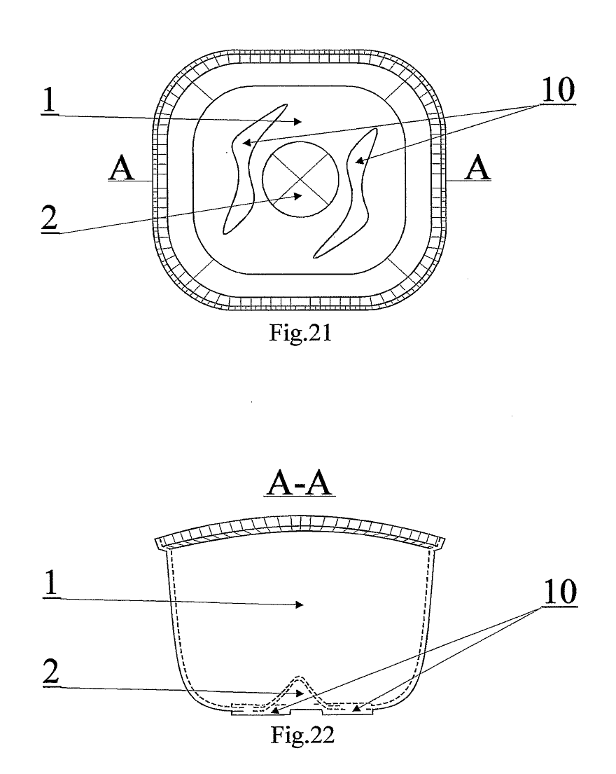

FIG. 21 top view of the container in its eighth embodiment, showing additionally formed swellings of the material in the form of patterns made in the container bottom on its outer side and on its inner side,

FIG. 22 section in A-A plane indicated in FIG. 21,

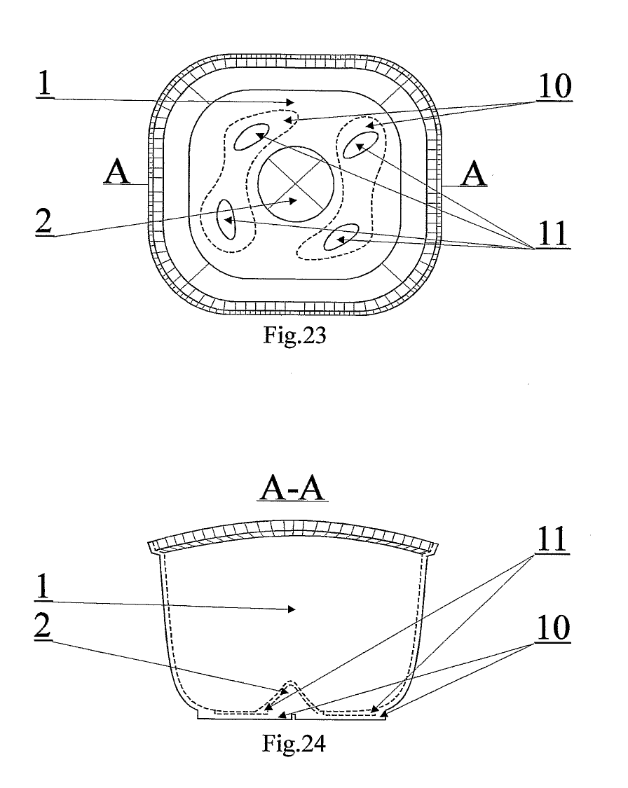

FIG. 23 top view of the container in its ninth embodiment, showing that in the swellings of the material formed in the container bottom on the outside, on the inside there are grooves,

FIG. 24 section in A-A plane indicated in FIG. 23,

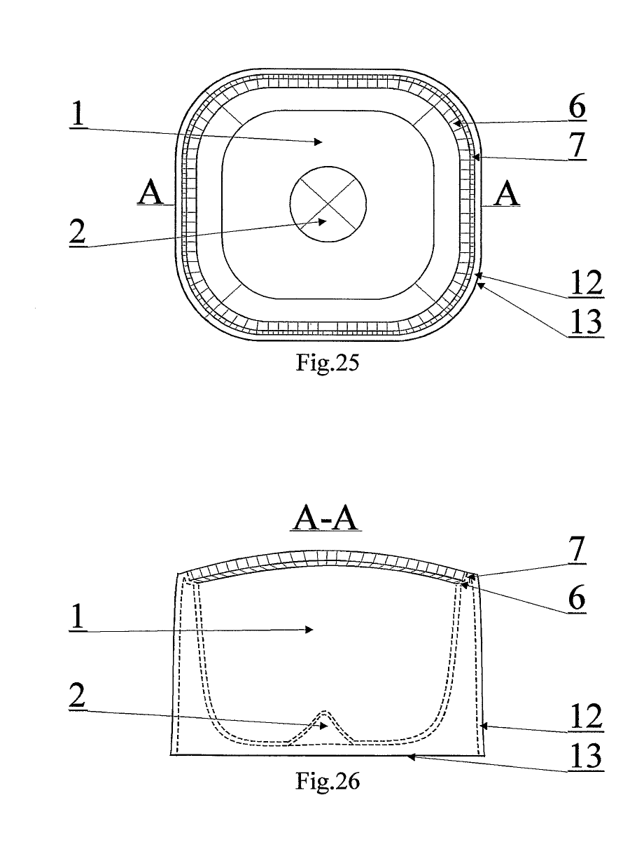

FIG. 25 top view of the container in its tenth embodiment, with an additional outer wall made from the rim deflected outwards, on which the container is placed,

FIG. 26 section in A-A plane indicated in FIG. 25,

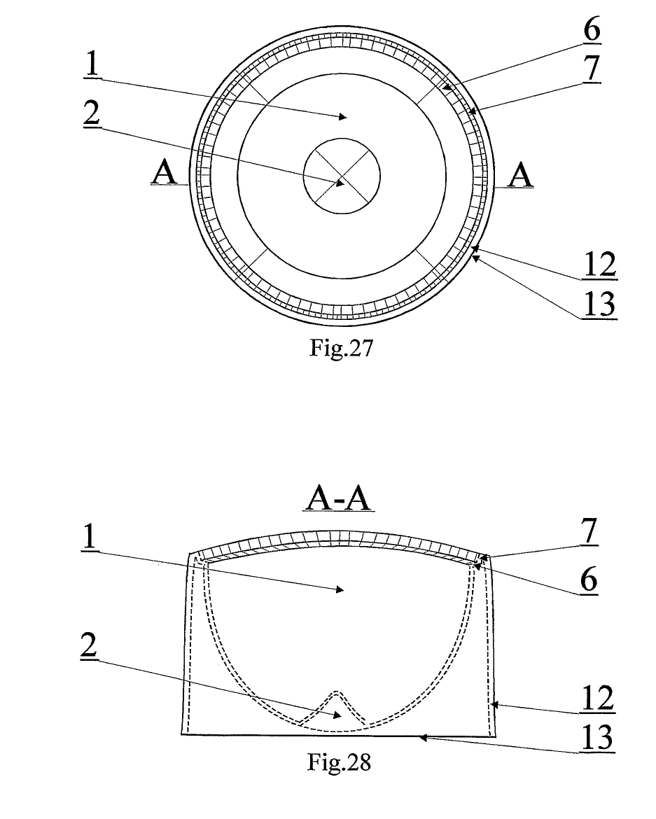

FIG. 27 top view of the container in its eleventh embodiment which has the shape of a hemisphere, placed on its outer wall,

FIG. 28 section in A-A plane indicated in FIG. 27,

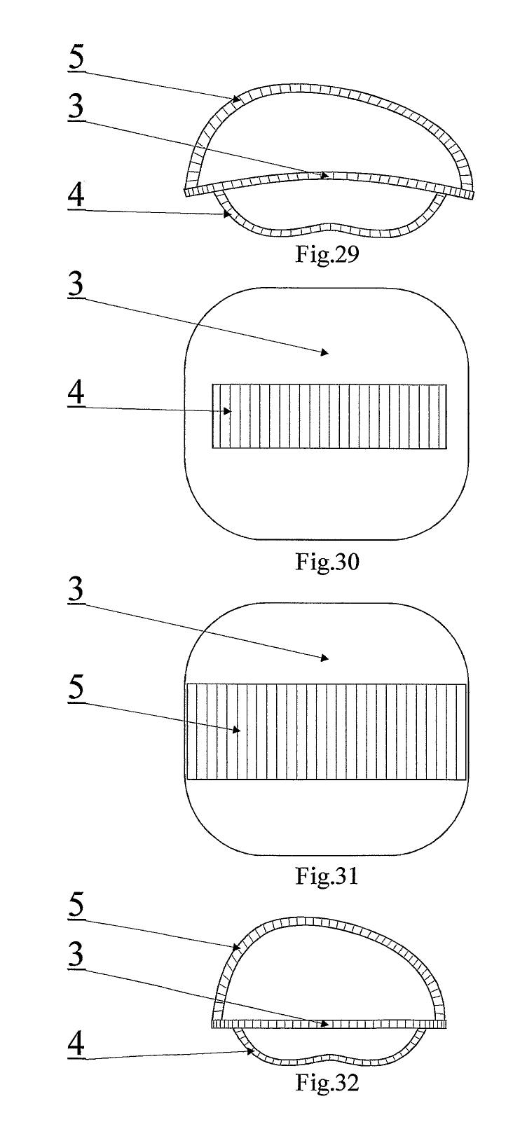

FIG. 29 side view of the lid in its first embodiment showing that the lid is arched,

FIG. 30 bottom view of the lid with the ice-fixing element, as shown in FIG. 29,

FIG. 31 top view of the lid with the handle, as shown in FIG. 29,

FIG. 32 side view of the lid in its second embodiment, showing that the lid is flat,

FIG. 33 side view of the lid in its third embodiment, with an at least double ice-fixing element in the form of an arched flat bar across which openings are made,

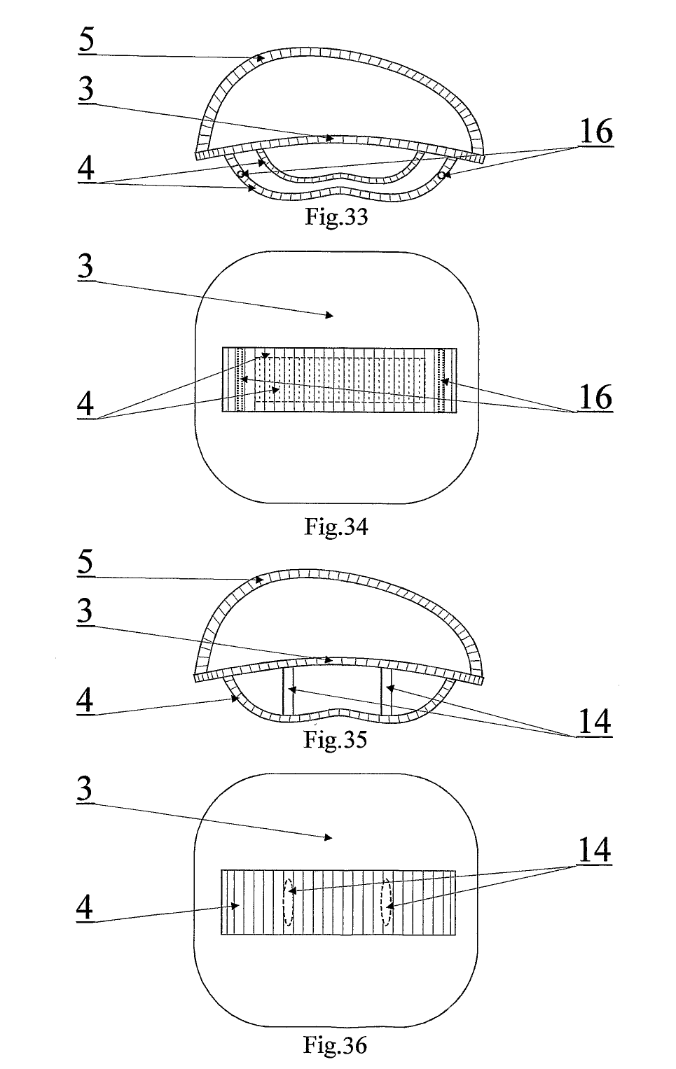

FIG. 34 bottom view of the lid shown in FIG. 33

FIG. 35 side view of the lid in its fourth embodiment, with the ice-fixing element in the form of an arched flat bar additionally attached to the lid bottom by appendages,

FIG. 36 bottom view of the lid as shown in FIG. 35

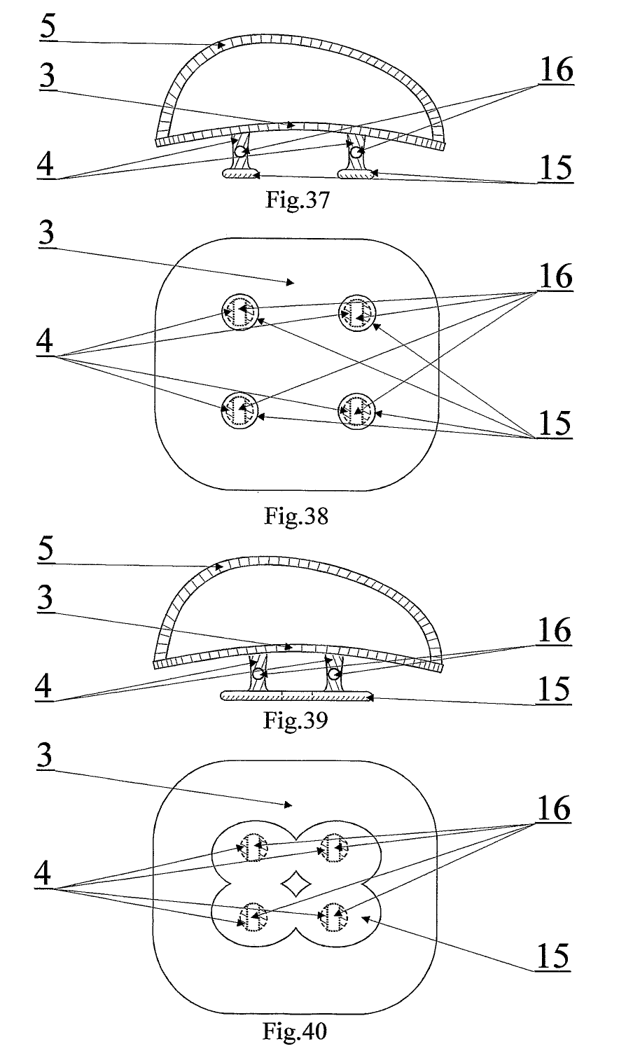

FIG. 37 side view of the lid in its fifth embodiment, with the ice-fixing element consisting of several appendages ending with brims, across which openings have been made,

FIG. 38 bottom view of the lid shown in FIG. 37

FIG. 39 side view of the lid in its sixth embodiment, with the ice-fixing element consisting of several appendages ending with interconnected brims, across which openings have been made,

FIG. 40 bottom view of the lid shown in FIG. 39

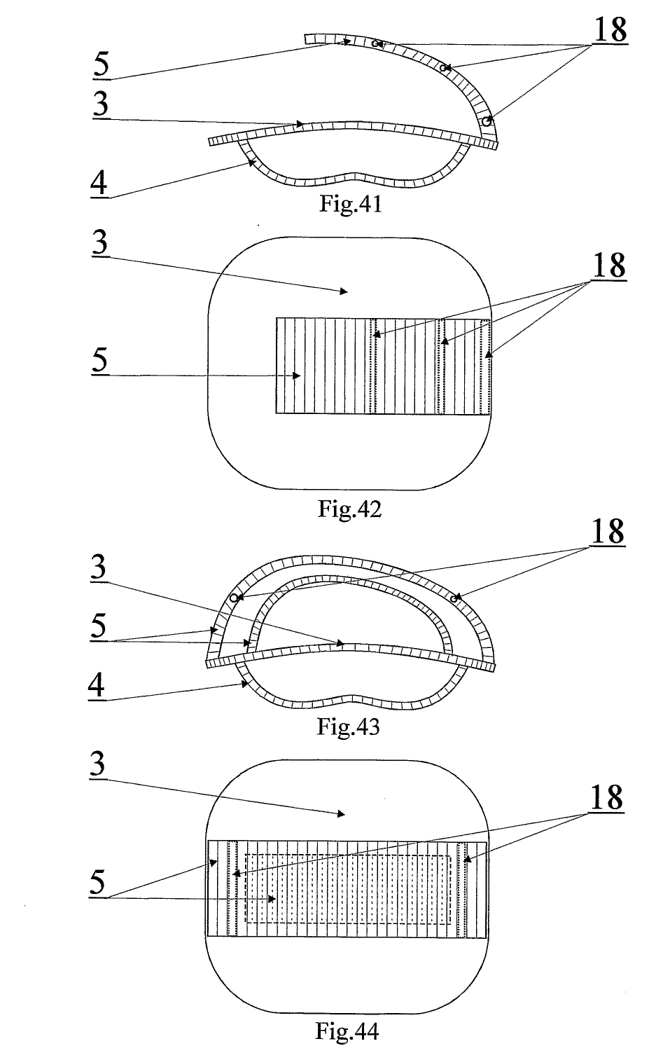

FIG. 41 side view of the lid in its seventh embodiment with a handle in the form of a quarter of an oval across which there are openings,

FIG. 42 top view of the lid shown in FIG. 41

FIG. 43 side view of the lid in its eighth embodiment, with at least a double handle,

FIG. 44 top view of the lid shown in FIG. 43

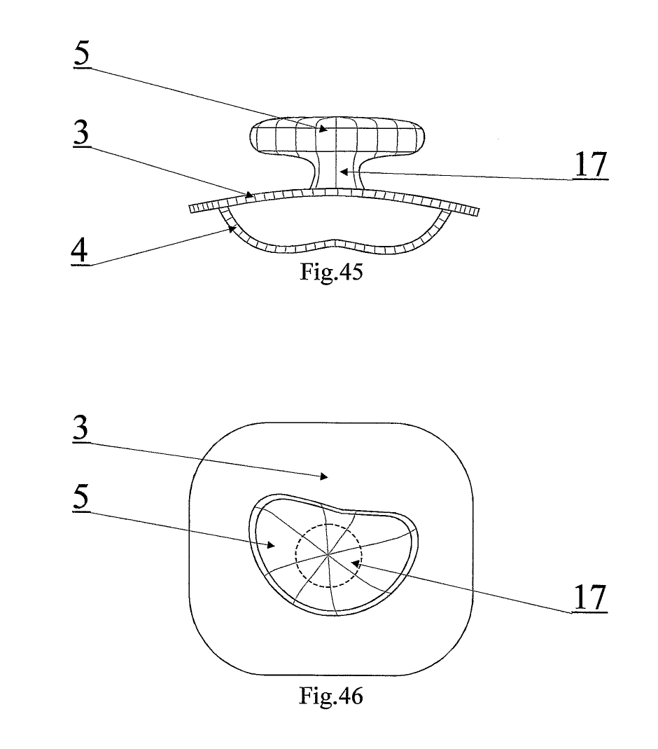

FIG. 45 side view of the lid in its ninth embodiment with the handle set on a core,

FIG. 46 top view of the lid shown in FIG. 45.

The inventive ice massage device consists of its lower part which is a container 1 open at the top, in whose bottom a conical cavity 2 is formed, and of a lid 3 together with an ice-fixing element 4 and a handle 5; the lid 3, the ice-fixing element 4 and the handle 5 making a uniform whole.

To prepare it for the massage, the container 1 is filled with water and then it is closed at the top with the lid 3 whereupon it is placed in a freezer. After the container 1 has been covered with the lid 3 the ice-fixing element 4 is submerged in water filled into container 1, which causes that when water freezes the lid 3 is linked together with the massaging ice block formed by the container 1.

In order to perform a massage, ice attached to the element 4 that fixes ice to the lid 3 is taken out of the container 1. If an ice layer is stuck to the inside of the container 1, then warm water is poured on the bottom of the container 1 from below for a few seconds.

In the process of forming a uniform ice block that precedes the massage treatment, freezing water increases its volume and deforms the form of the device pushing the lid 3 above the edge 6 of the container 1. Two solutions have been applied to solve this problem.

In the first embodiment of the device the first solution to the above problem has been applied by arching the edge 6 of the container 1 at the diametrically opposed sides and arching the lid 3 resting on the edge 6 of the container 1, which leads to the formation of an empty space between the surface of water and the lid 3 which allows the collection of the increased volume of freezing ice in that space. The arched lid 3 is protected against sliding off the edge 6 of the container 1 by the rim 7. The circumference of the edge 8 of the lid 3 fits the rim 7 of the edge 6 of the container 1 in such way that it adjoins it—FIG. 2, FIG. 3.

In the second embodiment of the device the second solution to the above problem has been applied by using a flat surface of the lid 3 and flat edges 6 of the container 1 in such way that the circumference of the edge 8 of the lid 3 is smaller than the edge 6 of the container 1, which causes that the lid 3 does not rest on the edge 6 of the container 1, but it is placed directly on the surface of water in the container 1, owing to which freezing ice raises it parallel to the position when increasing its volume —FIG. 4, FIG. 5.

The ice massage device is also intended for use in variable-temperature therapy, that is during a warm bath or shower which accelerate the ice melting process. That is why a uniform shape of the container 1 without a division into chambers was used, which causes that the ice block is compact and thereby has a bigger volume and in consequence a longer melting time, which results in a longer massage time.

In the first and second embodiment the container 1 has the shape of a polyhedron with a polygon base, whose walls join the base at an angle of not less than 90°. The joints of the container 1 walls are semicircular FIG. 6, FIG. 7, FIG. 8, FIG. 9.

In the third embodiment of the container 1 the edges of interconnected walls of the container 1 are sharp —FIG. 10, FIG. 11.

In the fourth embodiment the container 1 has the form of a hemisphere truncated at the bottom —FIG. 12, FIG. 13.

In the fifth embodiment of the container 1 the walls and the base of the container 1 have a stepwise structure consisting of interconnected rings 9 whose circumferences decrease towards the bottom and which are arranged on a transverse plane, parallel in relation to one another and perpendicularly to the basis. The stepwise structure permits the overlapping of the interconnected rings 9 in such way that rings of smaller circumferences are placed inside rings of bigger circumferences, which allows the container to be folded —FIG. 14, FIG. 15, FIG. 16.

In the sixth embodiment the container 1 in its cross-section has the shape of a closed figure with an irregular circumference —FIG. 17, FIG. 18.

The uniform shape of the container 1 without a division into chambers creates the problem of deformation of the container 1 bottom by the growing ice volume. This happens because the stress forces acting from the expanding ice act perpendicularly to the given point on the surface, therefore the bigger the surface the bigger the deforming force.

In order to prevent the deformation of the container 1 bottom a disruption in its uniform surface was made by forming a cavity 2 in the form of a cone, a stalagmite, in the bottom of the container 1, which resulted in a decrease in the surface-deforming force produced by freezing ice in proportion to the area of the uniform surface.

Additionally the surface of the bottom is reinforced by swellings of material 10 formed in the bottom, on which swellings the container 1 stands and which isolate the container 1 bottom from the freezer bottom. The isolation of the container 1 bottom from the bottom of the freezer is important since the water freezing process starts at the place of contact with the coldest element of the environment, that is the bottom of the freezer, where ice increases its volume the most.

In the seventh embodiment of the container 1, in the bottom on the outer part of the container 1 there are formed additional swellings of material 10 in the form of patterns on which the container rests —FIG. 19, FIG. 20.

In the eighth embodiment of the container 1, the formed additional swellings of material 10 in the form of patterns are located in the bottom on its outer side and on its inner side —FIG. 21, FIG. 22.

In the ninth embodiment of the container 1, in the swellings of material 10 made in the container 1 bottom on the outside, there are grooves 11 on the inner side of the container 1—FIG. 23, FIG. 24.

The most effective method for isolating the bottom of the container 1 is hanging it above the bottom of the freezer.

In the tenth embodiment of the container 1, in order to isolate the surface of the bottom of the container 1 from the bottom of the freezer, the rim 7 of the edge 6 of the container 1 wall is deflected outside in such way that it forms an additional outer wall 12 of the container 1, on whose edge 13 the container 1 stands —FIG. 25, FIG. 26.

In the eleventh embodiment of the container 1, the container 1 when put on the outer wall 12 has the form of a hemisphere, which is the best method for protecting the bottom of the container 1 against deformations by stresses from expanding ice —FIG. 27, FIG. 28.

In the first embodiment the lid 3 is arched and adjusted to the shape of the edge 6 of the container 1 upon which it rests. The ice-fixing element 4 is attached to the bottom of the lid 3 and has the shape of an arched flat bar deformed in the middle of the arch in the opposite direction. The deformation in the middle of the arch in the opposite direction is useful because it anchors ice in a more stable way and increases the proportion of ice quantity in the block used for the massage, which allows the massage to last longer —FIG. 29, FIG. 30, FIG. 31.

In the second embodiment the lid 3 is flat which allows it to be put directly on the surface of water. The placement of the flat surface of the lid 3 on the surface of water is advantageous because when the water has frozen the ice-fixing element 4 is wholly anchored in ice which causes that ice is more stably attached to the lid 3, which increases the feeling of comfort of the massage —FIG. 32.

Due to the weight of the anchored ice block and due to the resulting forces acting on the ice-fixing element 4 which can be damaged by these forces it is advisable to use solutions which will make it possible to distribute the weight of the ice block on a bigger number of edges and thus reinforce the structure of the element.

In the third embodiment of the lid 3, the ice-fixing element 4 having the shape of an arched flat bar is at least double —FIG. 33, FIG. 34.

In the fourth embodiment of the lid 3, the ice-fixing element 4 is additionally fixed to the bottom of the lid 3 by appendages 14 —FIG. 35, FIG. 36.

In the fifth embodiment of the lid 3, the ice-fixing element 4 consists of several appendages ending with brims 15 resembling classic anchors. To avoid the weight of ice resting only on the brims 15 of the appendages, above the brims 15, ports 16, on which the weight of ice is also supported, were situated transversely to the appendages —FIG. 37, FIG. 38.

In the sixth embodiment of the lid 3, in the ice-fixing element 4, the brims 15 are connected with one another —FIG. 39, FIG. 40.

The use of the ports 16 is justified for each of the presented solutions concerning the ice-fixing element 4, especially if the ports 16 are arranged in the points of a surface which is perpendicular to the surface of the lid 3 which causes that the weight of ice rests on the edges reinforced by the direction of fixing opposite to the direction of action of the force exerted by the weight of ice.

In the seventh embodiment of the lid 3, the handle 5 is attached only on one side to the top of the lid 3 and has the shape of a quarter of an oval. Across the handle 5 there are openings 18 which provide reinforcement of the structure in accordance with the rule that an element is stronger if the force acting on it can be spread on a larger number of edges —FIG. 41, FIG. 42.

In the eighth embodiment of the lid 3, the handle 5 is at least double —FIG. 43, FIG. 44.

In the ninth embodiment of the lid 3, the handle 5 is fixed on the core 17 —FIG. 45, FIG. 46.

1. An ice massage device comprised of a container, the container lid, the lid handle and an element fixing ice to the lid characterised in that the lid [3], the lid handle [5] and the ice-fixing element [4] make one whole, while in the bottom of the uniform, open-at-the-top container [1] there is a cavity [2] in the shape of a cone and directed towards the inside of the container.

2. A device as per claim 1, characterised in that the edge [6] of the container [1] and the lid are arched.

3. A device as per claim 2, characterised in that the edge [6] of the container [1] has a rim protecting the lid [3] from sliding off the edge [6] of the container [1].

4. A device as per claim 3. characterised in that the edge [8] of the lid [3] adjoins the rim [7] of the edge [6] of the container [1].

5. A device as per claim 1, characterised in that the edge [6] of the container [1] and the lid [3] are flat.

6. A device as per claim 5, characterised in that the circumference of the edge [8] of the flat lid [3] is smaller than the circumference of the flat edge [6] of the container [1].

7. A device as per claim 1, characterised in that the container [1] has the shape of a poly-hedron with a polygonal base, whose walls join the base at an angle of not less than 90°.

8. A device as per claim 7, characterised in that the joints of the container [1] walls are semicircular.

9. A device as per claim 7, characterised in that the edges of the interconnected walls of the container [1] are sharp.

10. A device as per claim 1, characterised in that the container [1] has the shape of a hemisphere truncated at the bottom.

11. A device as per claim 1, characterised in that the walls and the base of the container [1] have a stepwise structure consisting of interconnected rings [9] whose circumferences decrease towards the bottom and are arranged on a transverse plane parallel in relation to one another and perpendicularly to the basis.

12. A device as per claim 11, characterised in that the stepwise structure permits the overlapping of the interconnected rings [9] in such way that rings of smaller circumferences are placed inside rings of bigger circumferences, which allows the container [1] to be folded.

13. A device as per claim 1, characterised in that the container [1] in its cross-section has the shape of a closed figure with an irregular circumference.

14. A device as per claim 1, characterised in that in the bottom on the outer part of the container [1] there are formed additional swellings of material [10] in the form of patterns on which the container rests.

15. A device as per claim 14, characterised in that the formed additional swellings of material [10] in the form of patterns are located in the bottom on the outer and inner sides of the container [1].

16. A device as per claim 14, characterised in that in the swellings of material [10] made in the container [1] bottom on the outside, on the inner side of the container [1] there are grooves [11].

17. A device as per claim 1, characterised in that the rim [7] of the edge [6] of the wall of the container [1] is deflected outside forming an additional outer wall [12] of the container [1], on whose edge [13] the container [1] is placed.

18. A device as per claim 17, characterised in that the container [1] put up on the outer wall [12] has the shape of a hemisphere.

19. A device as per claim 1, characterised in that the ice-fixing element [4] has the shape of one arched flat bar slightly deformed in the middle.

20. A device as per claim 19. characterised in that the ice-fixing element [4] is at least double.

21. A device as per claim 19 or 20, characterised in that the ice-fixing element [4] is additionally attached to the bottom of the lid by appendages [14].

22. A device as per claim 1, characterised in that the ice-fixing element [4] consists of several appendages ending with brims [15].

23. A device as per claim 22, characterised in that in the ice-fixing element [4] the brims [15] are connected with one another.

24. A device as per claim 19 or 20 or 22 or 23, characterised in that in the ice-fixing element [4] there are openings [16] running across the ice-fixing element.

25. A device as per claim 1, characterised in that the handle [5] of the lid [3] is attached at its both ends to the top part of the lid [3].

26. A device as per claim 1, characterised in that the handle [5] of the lid [3] is attached at only one its end to the top part of the lid [3] and has the shape of a quarter of an oval.

27. A device as per claim 25 or 26, characterised in that the handle [5] of the lid [3] is at least double.

28. A device as per claim 1, characterised in that the handle [5] is fixed on a core [17].

29. A device as per claim 25 or 26 or 28, characterised in that in the handle [5] of the lid [3] there are openings [18] running across the handle.
